(19) United States
(12) Patent Application Publication
(10) Pub. No.: US 2002/0187250 A1
(43) Pub. Date: Dec. 12, 2002

(54) Anti-thrombogenic material and manufacturing method therefor

(75) Inventors: Tadashi Kokubo, Nagaokakyo-Shi (JP); Kim Monbin, Kyoto-Shi (JP); Kazuaki Muramatsu, Gamoumachi (JP); Akira Fujisawa, Kyoto-Shi (JP)

Correspondence Address: LAW OFFICE OF BARRY R LIPSITZ, 755 MAIN STREET, MONROE, CT, 06468, US

(73) Assignee: Miwatec Incorporated, Kawasaki-Shi (JP)

(21) Appl. No.: 10/214,923

(22) Filed: Aug. 7, 2002

Related U.S. Application Data
(63) Continuation of application No. 09/907,063, filed on Jul. 17, 2001.

(30) Foreign Application Priority Data
Jul. 21, 2000 (JP) 2000-220930

(51) Int. Cl. A61L 2/00; B05D 3/10

(52) U.S. Cl. 427/2.1; 427/243; 427/343; 427/2.24

(57) ABSTRACT

The invention provides an anti-thrombogenic material and methods for manufacturing anti-thrombogenic materials. The anti-thrombogenic material of the invention is particularly suited for coating the surface substrate of medical devices which come into contact with blood and biomedical tissues. The surface of the substrate, which may be made of pure titanium or titanium alloy, is provided with a porous layer having an irregular pore structure made of alkaline titanate. With this structure, it is possible to suppress the formation of fibrin induced by activation of the blood coagulation factor such as fibrinogen on the blood contact surface by coating the surface with alkaline titanate, and also to suppress the adhesion and activation of platelets. Moreover, since titanium and titanium alloy are inert for a living body and have favorable familiarity with it and also have large strength, the materials can be used to produce anti-thrombogenic medical devices of an implantable type.

The present application is a continuation of U.S. patent application Ser. No. 09/907,063 filed on Jul. 17, 2001, which claims the benefit of Japanese patent application number 2000-220930 filed on Jul. 21, 2000, the entire disclosures of which are incorporated herein by reference.

### TECHNICAL FIELD OF THE INVENTION

The present invention relates to an anti-thrombogenic material which is used for medical devices such as a blood pump for an artificial (auxiliary) heart, a prosthetic valve, a stent, a pacemaker, or the like. These devices have a surface which comes in contact with blood and biomedical tissues (referred to herein as a “blood contact surface”). The anti-thrombogenic material of the present invention is used to coat the blood contact surface of such medical devices. The invention also provides a manufacturing method for the anti-thrombogenic material.

### BACKGROUND OF THE INVENTION

The blood contact surface of the above-mentioned medical devices will obstruct blood flow and may harm a human body in the event that blood components are brought into contact with the blood contact surface of such devices and the blood components are formed into thrombi. Therefore, it is important to make the above-mentioned blood contact surface of such medical devices resistant to forming thrombi, and for this purpose, a mirror-polished surface has conventionally been desired so that thrombi are not caused by stagnation and turbulent flow of blood.

For this requirement, the Japanese Patent Laid-Open 6-296682 (296682/1994) offers an improvement in anti-thrombogenic property by forming a ceramic coating film, which has somewhat more rough surface than a mirror-polished surface, on the substrate by using thin film deposition by a sputtering method.

This prior art is based on the following technological concept. Namely, the fact that platelets are activated and adhere to a surface of a material which comes into contact with blood is caused by the fact that only particular proteins are aggregated, when various proteins (hereinafter called membrane proteins) adhering to the surface of the platelets almost uniformly distributed in the blood are brought into contact with proteins adsorbed on the surface of the above blood contact material. Here, the particular proteins are such proteins as are contained in the above-mentioned membrane proteins and tend to aggregate dependent on each material of the blood contact surface. Therefore, when surface roughness Rmax in a minute area of 500 nm is made to 10 nm or larger, namely, when it is made larger than the size corresponding to that of the membrane proteins, it becomes difficult for the membrane proteins to come into contact with the deep recessed parts in the rough blood contact surface. Therefore, it is difficult for the above-mentioned particular proteins to aggregate on the blood contact surface. As the result, the platelets become resistant to adhering on the surface.

The problem of the above prior art is the fact that it is difficult to form a film on the blood contact surface of complex shapes and moreover, an expensive sputtering thin film deposition device is necessary.

Namely, according to said prior art, a dry process such as film formation by a vapor growth method and a sputtering method has been used as a method capable of controlling the surface roughness in a minute area of 500 nm. However, in the dry process, it has been difficult to surely form a film, for example, on the inner wall of a largely curved cylindrical body.

Moreover, another problem of the above prior art is the fact that although there have been two kinds of thrombus-forming reactions such as (1) formation of fibrin by activation of a blood coagulation factor such as fibrinogen on the blood contact surface and (2) adhesion and activation of platelets, the conventional art has not been effective against the phenomenon of (1), and a sufficient anti-thrombogenic property has not been achieved.

The purpose of this invention is to solve the problems included in such a conventional constitution, and to provide an anti-thrombogenic material and a manufacturing method thereof by which surface treatment is secured even onto the blood contact surface of complex shapes, and moreover, not only the formation of fibrin on the blood contact surface caused by activation of the blood coagulation factor such as fibrinogen, but also both of the adhesion and activation of platelets can be suppressed.

### SUMMARY OF THE INVENTION

As a result of keen examination by the inventors to solve the above problems, the invention has been made by discovering that, in order to suppress the formation of fibrin caused by activation of the blood coagulation factor such as fibrinogen on the blood contact surface, selection of a material is rather more important than a shape of the blood contact surface, and that alkaline titanate possesses not only a property to suppress the formation of fibrin by activation of the blood coagulation factor such as fibrinogen on the blood contact surface, but also a property to suppress the adhesion and activation of platelets at the same time.

Namely, the anti-thrombogenic material of the invention is characterized in that the surface of the substrate made of pure titanium or titanium alloy is provided with a porous layer having an irregular pore structure made of alkaline titanate.

With this structure, it is possible to suppress the formation of fibrin induced by activation of the blood coagulation factor such as fibrinogen on the blood contact surface by coating on the surface with alkaline titanate, and also to suppress the adhesion and activation of platelets. Moreover, since titanium and titanium alloy are inert for a living body and have favorable familiarity with it and also have large strength, the materials can be used to produce anti-thrombogenic medical devices of an implantable type.

The alkaline titanates are the chemical compounds expressed by HTiO₃⁻.nH₂O+R⁺ (R is an alkaline metal or an alkaline earth metal)=RHTiO₃.nH₂O.

Further, since the surface of the anti-thrombogenic material is made from a porous layer and the area of the substrate surface becomes smaller than a smoothed surface, and as a result, the contact area between the platelets and the substrate surface is decreased, the resistant action to the aggregation of the membrane proteins of the platelets is reinforced. Moreover, since the above porous layer is made to have an irregular pore structure, the platelets tend to adhere to the surface at unequal intervals due to the irregularity of the surface structure even if the platelets adhere to the surface. Therefore, chain overlapping of the platelets is apt to be immediately terminated, and as a result, the action for making the film proteins of the platelets resistant to aggregation is reinforced.

Moreover, the above alkaline titanate can be in any of gelatinous, amorphous, and crystalline states, as far as it has sufficient adhesion to the substrate at a level to be not separated from the substrate in the blood flow.

The porous layer surface may also be coated with a calcium phosphate material on the surface of the porous layer.

Since this structure is coated with a calcium phosphate material on the surface, albumin of plasma proteins is much adsorbed on this calcium phosphate material. The adsorption face of albumin exerts an excellent anti-thrombogenic property.

The pore size of the porous layer may be smaller than 1 μm on average.

Since the platelets range in size from 1 to 3 μm, it is possible, by using this structure, to effectively prevent the above particular proteins from staying in the pores and aggregating therein by making the average pore diameter smaller than that of these platelets.

The anti-thrombogenic materials of the invention can be manufactured by the manufacturing methods described below.

The manufacturing method is characterized in that the substrate made of pure titanium or a titanium alloy is immersed in an alkaline solution and thereby a porous layer having an irregular pore structure made of alkaline titanate is formed on the surface of the pure titanium or titanium alloy substrate.

According to this structure, a porous gelatinous layer having an irregular pore structure of an alkaline titanate is formed on the substrate surface by immersing the pure titanium or titanium alloy substrate in the alkaline solution. Since this method does not use a dry process but uses an immersion method, it is possible to surely form an anti-thrombogenic surface by this method even if the blood contact surface is in complex shapes. Moreover, this method does not need an expensive thermal spraying equipment.

Moreover, the alkaline solution stated above comprises a solution containing ions of an alkaline metal or an alkaline earth metal, preferably, an aqueous solution containing one or more kinds of the ions of sodium (Na⁺), kalium (K⁺), and calcium (Ca²⁺). Moreover, the alkaline solution treatment can be carried out in a molar concentration from 0.1 to 15 mol, at temperatures of 10 to 95° C., and with a reaction time for an hour to one week.

The porous layer of alkaline titanate is formed by the following mechanism.

Pure titanium and a titanium alloy has a coating of titanium oxide on the surface, and this titanium oxide dissolves in the alkaline aqueous solution, and a reaction of erosion takes place on the metal surface in accordance with the following mechanism, as a result, the porous layer is formed.

TiO₂+OH⁻=HTiO₃⁻ (1)

Ti+3OH=Ti(OH)₃⁻+4e (2)

Ti(OH)₃⁺+e⁻=TiO₂.H₂O−1/2H₃ (3)

Ti(OH)₃⁺+OH=Ti(OH)₄ (4)

TiO₂.nH₂O+OH⁻=HTiO₃.nH₂O (5)

HYiO₃⁻.nH₂O+R⁺ (R is an alkaline metal or an alkaline earth metal)=RHTiO₃.nH₂O (6)

Next, the manufacturing method may further include heat treating the substrate. In particular, after the substrate is immersed in the alkaline solution as described above, the substrate is further heat-treated at the transition temperature of titanium of 882° C. or lower so as not to be deteriorated in strength.

With this structure, it is possible to make the alkaline titanate to be amorphous or crystallized. In this case, oxygen is diffused, and as a result, lots of titanium oxide phases come into existence in the interface part of the porous layer across the substrate.

Moreover, since the above porous layer is provided with lots of titanium oxide phases in the interface part across the substrate by the heat treatment as described above and the pure titanium or the titanium alloy constituting the substrate is of the same system material, the bonding strength is large, and as a result, it is possible to increase the bonding strength to the substrate.

Here, the heat treatment can be carried out for a heating time of 1 to 24 hours at temperatures of 300 to 800° C. in an atmospheric oven.

Next, the manufacturing method may be further characterized in that after the substrate is immersed in the alkaline solution or after the substrate is heated at a temperature below the transition temperature of titanium as discussed above, calcium phosphate is made to be precipitated on said porous layer by further immersing the substrate in a pseudo body fluid.

According to this structure, it is possible to easily manufacture the anti-thrombogenic material described above.

Moreover, the above calcium phosphate material is formed by the following mechanism.

By immersing alkaline titanate in a pseudo body fluid (pH=7.0-7.5) containing calcium and phosphorus, alkaline metal ions and alkaline earth metal ions like Na⁺ etc. are emitted from the fluid in this environment, and a Ti—OH group is formed on the surface of the alkaline titanate by incorporating H₃O⁺ ions therein instead. This Ti—OH group induces nucleus formation of the calcium phosphate material, and the formed calcium phosphate material grows by incorporating calcium ions and phosphoric acid ions from the ambient fluid.

The above pseudo body fluid means an aqueous solution which imitates ion components contained in human plasma components and contains those ions of Na⁺, K⁺, Mg²⁺, Ca²⁺, Cl⁻, HCO³⁻, HPO₄²⁻, and SO₄²⁻. Moreover, it is possible to control a composition ratio of each element in the calcium phosphate formed on the surface by arbitrarily varying each ion concentration contained in this pseudo body fluid.

Here, the immersion treatment in the pseudo body fluid can be carried out in a reaction time within four weeks and at temperatures of 10.0 to 99.9° C.

### DETAILED DESCRIPTION OF EXEMPLARY EMBODIMENTS

This invention will be explained below based on the embodiments, but the invention is not to be restricted to the embodiments.

Embodiment 1

(Surface Treatment of Bio-Material)

As a pretreatment, a mirror-finished pure titanium material (size: 10×10×1 mm, Ra=0.7 nm [per 10×10 μm]) was ultrasonically cleaned in toluene, and rinsed with ethanol and distilled water. After this titanium material was immersed in 5M NaOH for 24 hours at 60° C., the surface was rinsed with distilled water and dried for 24 hours at 40° C.

Following the above, the titanium material was heat-treated at 600° C. for one hour (temperature rising rate=5° C./min).

From a surface image through a SEM, it was confirmed that a porous layer with an irregular pore structure having pores of an average diameter smaller than 1 μm was formed on the article of the embodiment 1. Moreover, it was confirmed by thin film X-ray diffraction that the porous layer was an amorphous layer, and it was further confirmed from the variation in Auger electron peak in the direction of the depth that the porous layer was alkaline titanate having an inclined structure in which the electron peak was gradually decreasing toward the inside of the metal.

### EVALUATION EXAMPLE

### Evaluation of Blood Compatibility

The titanium material surface-treated in accordance with the embodiment 1 was sterilized in an autoclave (121° C., 20 minutes). This sample was warmed in a 37° C. physiological salt solution for 10 minutes in advance and thereafter it was incubated for one hour in heparinized human fresh blood (final concentration of heparin: 1.0 lU/ml) kept at 37° C. After the incubation, the sample was taken out of the blood and the surface was cleaned with a physiological salt solution three times. Following this, the surface of the sample was fixedly treated with a physiological salt solution containing 2.5% glutaraldehyde for 20 minutes at a room temperature. After the fixation was completed, the surface of the sample was cleaned with a physiological salt solution three times and further rinsed with distilled water two times, and then it was lyophilized.

(Evaluation Result)

Concerning the article of the embodiment 1, adhesion of platelets and formation of protein fiber consisting of fibrin were not observed through the observation by a SEM.

Embodiment 2

The article of the embodiment 1 was further immersed in a 36.5° C. pseudo body fluid for one hour (pH=7.40, the respective ion concentrations (mM) contained in the components are Na+: 142.0, K⁺: 5.0, Ca²⁺: 2.5, Mg²⁺: 1.5, Cl: 147.8, HCO₃⁻: 4.2, HPO₄²⁻: 1.0, and SO₄²⁻: 0.5). Finally, the surface was rinsed with distilled water and dried.

By means of thin film X-ray diffraction, it was confirmed that the article of the embodiment 2 immersed in the pseudo body fluid was coated with the amorphous layer and it was further confirmed from the variation in Auger electron peak in the direction of the depth that the layer was an alkaline titanate having an inclined structure in which the electron peak was gradually decreasing toward the inside of the metal, and that apatite, a kind of calcium phosphate material, was formed on the surface of the porous layer.

As a result of an evaluation of blood compatibility similar to the case of the embodiment 1, adhesion of platelets and protein fiber of fibrin was not observed at all on the article of the embodiment 2 from the observation through the SEM.

### Comparison Example 1

As a comparison example 1, an evaluation of blood compatibility similar to the case of the embodiment 1 was performed by using a piece of mirror-finished pure titanium (surface roughness Ra=0.7 nm per 10×10 μm).

As a result, it was found that a lot of protein fiber of fibrin adheres to the surface and that thrombi were caused by aggregation and adhesion of platelets and red blood corpuscles. These protein fiber of fibrin, platelets, and red blood corpuscles covered 51% of the blood contact surface.

As a comparison example 2, an evaluation of blood compatibility similar to the case of the embodiment 1 was performed by using a piece of pure titanium (surface roughness Ra=42.6 nm, 10×10 μm) polished with a sheet of #800 waterproof abrasive paper.

As a result, it was found that a lot of protein fiber of fibrin adheres to the surface and that thrombi were caused by aggregation and adhesion of platelets and red blood corpuscles. These protein fiber of fibrin, platelets, and red blood corpuscles covered 68% of the blood contact surface.

Effects of the Invention

As described above, by using the anti-thrombogenic material in accordance with this invention, it is possible to suppress the formation of fibrin caused by activation of the blood coagulation factor such as fibrinogen on the blood contact surface by means of coating the substrate surface with alkaline titanate. Moreover, since titanium and titanium alloy constituting the substrate are inert toward a living body and have favorable familiarity with it and also have large strength, the materials can be applied to anti-thrombogenic medical devices of an implantable type.

Further, since the surface of this anti-thrombogenic material is made porous and the contact area between the platelets and the substrate surface is decreased, the action to make the membrane proteins of the platelets resistant to aggregation is reinforced. Moreover, since the above porous layer is made to have an irregular pore structure, the platelets tend to adhere to the surface at unequal intervals due to the irregularity of the surface structure even if the platelets adhere to the surface, therefore, chain overlapping of the platelets is apt to be easily ended, as a result, the action for making the membrane proteins of the platelets resistant to aggregation is reinforced.

Moreover, since the anti-thrombogenic material can be formed on the substrate surface by immersing the substrate in a specific solution by the manufacturing method in accordance with this invention, anti-thrombogenic surface can surely be formed even if the blood contact surface is in complex shapes. Further, the surface can be coated with calcium phosphate very easily which is effective as anti-thrombogenic property.

What is claimed is:

1. A method for preventing the formation of fibrin and the activation and adhesion of platelets on the surface of a medical device, comprising the steps of:
coating at least a portion of the medical device with a substrate comprised of one of titanium or titanium alloy; and
immersing the coated portion of the medical device in an alkaline solution to form a porous layer of alkaline titanate on the surface of said substrate, said porous layer having an irregular pore structure.

2. A method in accordance with claim 1, wherein said porous layer comprises a gelatinous layer.

3. A method in accordance with claim 1, wherein said medical device has a complex shape.

4. A method in accordance with claim 1, wherein said alkaline solution comprises an aqueous solution containing at least one of sodium ions, potassium ions, and calcium ions.

5. A method in accordance with claim 4, wherein said molar concentration of said alkaline solution is between 0.1 to 15.0 mol.

6. A method in accordance with claim 4, wherein said alkaline solution is heated to a temperature of between 10° and 95° C.

7. A method in accordance with claim 4, wherein said coated portion is immersed in said alkaline solution for a period of time between one hour to one week.

8. A method in accordance with claim 1, further comprising:
heating the substrate to a temperature not higher than 882° C. after said immersing step.

9. A method in accordance with claim 8, wherein said substrate is heated for a period of time between one to twenty-four hours at a temperature between 300° C. to 800° C.

10. A method in accordance with claim 8, further comprising:
precipitating calcium phosphate on said porous layer by immersing the coated portion of the medical device in a pseudo body fluid.

11. A method in accordance with claim 1, further comprising:
precipitating calcium phosphate on said porous layer by immersing the coated portion of the medical device in a pseudo body fluid.

12. A method in accordance with claim 11, wherein said pseudo body fluid has a pH of between 7.0 and 7.5.

13. A method in accordance with claim 1, wherein said medical device comprises a medical instrument.

14. A method in accordance with claim 1, wherein said medical device is an implantable device.

15. A method in accordance with claim 14, wherin said implantable device comprises one of a stent, a prosthetic valve, a blood pump, an artificial heart, or a pacemaker.

16. A method in accordance with claim 1, wherein a contact area of the porous layer is smaller than a surface area of the substrate.

17. A method in accordance with claim 1, wherein said layer of alkaline titanate exists in at least one of a gelatinous state, an amorphous state, or a crystalline state on the surface of said substrate.

18. A method in accordance with claim 1, wherein an average pore size of said porous layer is less than 1 μm.
